(19) United States
(12) Patent Application Publication
(10) Pub. No.: US 2022/0318596 A1
(43) Pub. Date: Oct. 6, 2022

(54) Learning Molecule Graphs Embedding Using Encoder-Decoder Architecture

(71) Applicant: Microsoft Technology Licensing, LLC, Redmond, WA (US)

(72) Inventors: Mohammad Reza SARSHOGH, Seattle, WA (US); Robin Abraham, Redmond, WA (US)

(21) Appl. No.: 17/219,257

(22) Filed: Mar. 31, 2021

(51) Int. Cl. G06N 3/04; G16C 20/70

(52) U.S. Cl. CPC G06N 3/0454; G16C 20/70

(57) ABSTRACT

An Encoder-Decoder architecture uses two neural networks that work together to learn molecule embedding without any labeled data by transform the molecule graph to an embedding, and then mapping that embedding to a character-based representation of that molecule. An encoder operates as a molecule embedding model to produce a vector of length “n” that reperesents the molecule as a point in an n-dimentional cartesian space. The generated vector is used by a decoder to predict the molecule's character-based representation such as a SMILES, only based on the molecule structure. A loss function is applied to the decoded character-based representation compared to the actual character-based representation of that molecyle, to generate a gradient of the error determined by the loss function which is used to modify weights in the encoder-decoder model during training.

### BACKGROUND

Measuring molecule properties and detecting similar molecules play a major role in drug discovery and development. Properties of a first molecule may be known. It may be desirable to identify other molecules that have properties similar to the properties the first molecule. But using a lab to identify molecules similar to known molecules based on some specific criteria is very expensive and time consuming And selecting which properties to measure may also be time consuming and expensive. Depending on the instrument and measurement procedure, there may be inconsistencies in measured data, which may affect the usability of the measured data. Furthermore, because of budgetary and time limitations, it may not be possible to measure selected properties on all eligible molecules.

### SUMMARY

An Encoder-Decoder architecture uses two neural networks that work together to transform a molecule graph to a character-based sequence. Mapping molecule graphs to an embedding space is performed using a Graph Neural Networks (GNNs) model as an encoder. The encoder operates as a molecule embedding model to produce a vector that is an n-dimensional representation of a point in the embedding space. The generated vector is used by a decoder to generate the molecule's character-based representation such as a simplified molecular-input line-entry system (SMILES) token or sequence, only based on the molecule 2D structure. A loss function is applied to the character-based representation compared to the generated squence by the decoder to calculate a gradient of the error which is porpageted through the whole network and modifies the molecule embedding model and decoder during training.

### BRIEF DESCRIPTION OF THE DRAWINGS

FIG. 1 illustrates an example system for predicting a characteristic of a molecule.

FIG. 2 illustrates an example graph that includes nodes and edges.

FIG. 3A illustrates an example node embedding model that includes graph neural network layers and attention layers.

FIG. 3B illustrates neighboring nodes passing messages to a receiving node.

FIG. 4 illustrates an example node aggregation model.

FIG. 5 illustrates an example decoder in connection with an embedding model.

FIG. 6 is a flowchart of an example computer implemented method for determining embedded features of a node in a graph.

FIG. 7 is a flowchart of an example computer implemented method for determining graph features for a graph.

FIG. 8 is a flowchart of an example computer implemented method for training an embedding model using backpropagation from a decoder.

FIG. 9 illustrates certain components that can be included within a computing device.

### DETAILED DESCRIPTION

In the following description, reference is made to the accompanying drawings that form a part hereof, and in which is shown by way of illustration specific embodiments which may be practiced. These embodiments are described in sufficient detail to enable those skilled in the art to practice the invention, and it is to be understood that other embodiments may be utilized and that structural, logical and electrical changes may be made without departing from the scope of the present invention. The following description of example embodiments is, therefore, not to be taken in a limited sense, and the scope of the present invention is defined by the appended claims.

The functions or algorithms described herein may be implemented in software in one embodiment. The software may consist of computer executable instructions stored on computer readable media or computer readable storage device such as one or more non-transitory memories or other type of hardware-based storage devices, either local or networked. Further, such functions correspond to modules, which may be software, hardware, firmware or any combination thereof. Multiple functions may be performed in one or more modules as desired, and the embodiments described are merely examples. The software may be executed on a digital signal processor, ASIC, microprocessor, or other type of processor operating on a computer system, such as a personal computer, server or other computer system, turning such computer system into a specifically programmed machine.

The functionality can be configured to perform an operation using, for instance, software, hardware, firmware, or the like. For example, the phrase “configured to” can refer to a logic circuit structure of a hardware element that is to implement the associated functionality. The phrase “configured to” can also refer to a logic circuit structure of a hardware element that is to implement the coding design of associated functionality of firmware or software. The term “module” refers to a structural element that can be implemented using any suitable hardware (e.g., a processor, among others), software (e.g., an application, among others), firmware, or any combination of hardware, software, and firmware. The term, “logic” encompasses any functionality for performing a task. For instance, each operation illustrated in the flowcharts corresponds to logic for performing that operation. An operation can be performed using, software, hardware, firmware, or the like. The terms, “component,” “system,” and the like may refer to computer-related entities, hardware, and software in execution, firmware, or combination thereof. A component may be a process running on a processor, an object, an executable, a program, a function, a subroutine, a computer, or a combination of software and hardware. The term, “processor,” may refer to a hardware component, such as a processing unit of a computer system.

Furthermore, the claimed subject matter may be implemented as a method, apparatus, or article of manufacture using standard programming and engineering techniques to produce software, firmware, hardware, or any combination thereof to control a computing device to implement the disclosed subject matter. The term, “article of manufacture,” as used herein is intended to encompass a computer program accessible from any computer-readable storage device or media. Computer-readable storage media can include, but are not limited to, magnetic storage devices, e.g., hard disk, floppy disk, magnetic strips, optical disk, compact disk (CD), digital versatile disk (DVD), smart cards, flash memory devices, among others. In contrast, computer-readable media, i.e., not storage media, may additionally include communication media such as transmission media for wireless signals and the like.

Measuring molecule properties and detecting similar molecules may be important to drug discovery and development. Certain properties of a first molecule may be known. It may be desirable to identify other molecules that have properties similar to the certain properties of the first molecule. For example, a first molecule may be known to be effective for treating HIV, and it may be desirable to identify other molecules that have properties similar to the first molecule because such other molecules may also be effective for treating HIV. But identifying other molecules that have properties similar to the certain properties of the first molecule may be challenging. Identifying similar molecules may involve expensive and time-consuming laboratory work.

Selecting which properties of eligible molecules to measure may also be time consuming and expensive. Depending on the instrument and measurement procedure, there may be inconsistencies in measured data, which may affect the usability of the measured data. Furthermore, because of budgetary and time limitations, it may not be possible to measure selected properties on all the eligible molecules.

An Encoder-Decoder architecture uses two neural networks that work together to transform the molecule graph to a character-based sequence. Mapping molecule graphs to an embedding space is performed using Graph Neural Networks (GNNs). The GNNs operate as a molecule embedding model to produce a vector of size n that is the reperesentation of the molecule in an n-dimensional cartesian space. The generated vector is used by a decoder to generate the molecule's character-based representation such as a simplified molecular-input line-entry system (SMILES) sequence, only based on the molecule 2D structure. A loss function is applied to the character-based representation compared to the generated squence by the decoder to calculate the gradient of the error which is porpageted through the whole network and modifies the molecule embedding model during training.

Once the molecule embedding model is trained (encoder), the resulting embedding space can be used to find similar molecules. Also, the embedding model can be merged with another model to predict different properties of a molecule, the same way a pretrained ResNet, DenseNet, etc is used in computer vision models.

Mapping the molecule to the embedding space may allow efficient comparison of the molecule with another molecule (which may have certain known properties) that has been mapped to the embedding space. Mapping the molecule to the embedding space may also allow efficient predictions regarding whether the molecule will be effective for a particular task or will possess a particular property.

One way the embedding model may facilitate finding molecules with similar properties is through mapping molecules to the embedding space. Once the molecules are mapped to the embedding space, distances between the molecules can be calculated using co-sign or cartesian distance between two points in an n-dimensional cartesian space. When a first molecule is close to a second molecule in the embedding space (which may be referred to as neighboring molecules), the first molecule and the second molecule may have similar properties. Thus, if the first molecule has known properties, subsequent lab testing may focus on molecules that neighbor the first molecule to determine whether those neighboring molecules also have the known properties. Using the approach of identifying close molecules in the embedding space can reduce the search space considerably and consequently reduce the required time and expenses incurred in lab testing.

Another way the embedding model may facilitate identifying molecules with certain properties is through merging the embedding model with another model (such as a task-specific model) to learn how to predict different properties of a molecule (such as predicting whether a given molecule has toxic properties) even with a few labeled data during the training phase. This use of the embedding model may be similar to how pretrained ResNet and DenseNet models are used in connection with computer vision models.

By applying the encoder decoder approach to retrain the molecule embedding model (encoder), learned weights may be used to hot-start the training phase of different downstream prediction tasks, such as predicting binding results for a set of inhibitors of human β-secretase 1, or predicting octanol/water distribution coefficient (logD at pH 7.4). On several public datasets, an improvement in ROC-AUC (receiver operating characteristic area under the curve) classification metric, in classification tasks, and in RMSE (root mean square error) in regression tasks has been observed.

The generic embedding space can be used as a core of other models to improve the accuracy and training time. Also, it can be very helpful when there is insufficient access to labeled training data.

The graph representation of the molecule (which may be referred to as a molecule graph) may include a node (which may be referred to as a vertex) for each atom in the molecule and an edge (which may be referred to as a link) for each bond connecting atoms in the molecule. Each node in the graph and each edge in the graph may have features. The features may convey information regarding the node or the edge. Features of each node in the graph may be based on attributes and characteristics of each corresponding atom, such as atomic number, chirality, charge, etc. Features of each edge in the graph may be based on attributes and characteristics of each corresponding bond, such as bond type, bond direction, etc. The graph representation of the molecule may be based on a 2D structure of the molecule which can be deducted from molecule's SMILES. Other character-based representations may be used in further embodiments, but for convenience, description of various embodiments will reference SMILES representations. A SMILES may be a chemical notation that represents a chemical structure of a molecule in text or character-based way. Other representations may be used in further embodiments. An RDKit library may translate SMILES to molecule structure. The molecule structure generated by RDKit may be converted to a graph data structure that may be consumed by molecule embedding model as an input.

The embedding model may include a node to vector model (an atom embedding model), which may use graph neural networks to map each atom of a molecule to a feature space based on a molecule structure of the molecule. The embedding model may include an aggregation model that generates molecule features based on learned features of atoms in the molecule. The node to vector model may, using graph neural networks, generate embedded atom features (learned features) for each atom in the molecule. The aggregation model may generate embedded molecule features (learned features) for the molecule, based on the learned features of it's atoms. The learned features for the molecule may define a location of the molecule in an embedding space.

The atom embedding model may include an embedding layer and one or more graph neural network (GNN) layers. A GNN may be a type of neural network that operates directly on a graph structure. A GNN may follow a recursive neighborhood aggregation scheme.

The embedding layer may map an atomic number of each atom (which may be represented as a node in an input graph) to a denser feature space, which may help the embedding model learn a more accurate feature space for atoms. The embedding layer may map an atomic number of each node to a vector of a defined size using linear mapping and/or a lookup table. The embedding layer may be a standard way of moving from a discrete set of entities (such as atoms) to a denser space (such as a vector of size n). The vector associated with each atomic number plus other features of the atom may define updated features of the node. The atomic number and the other features of the atom may be input features of the node that represents the atom. The updated features of the node may be based on the input features of the node. The updated features of the node may be a singular representation that has all the information of the input features of the node embedded into it. The input features of the node may be based on attributes and characteristics of the node.

Each GNN layer in the one or more GNN layers may receive a molecule graph and determine embedded atom features for each atom in the molecule graph. The embedded atom features of an atom may convey specific information regarding the atom, its associated bonds, and a neighborhood of the atom. A first GNN layer in the one or more GNN layers may receive the input graph or the updated graph and determine first layer embedded atom features for each atom in the molecule. Each subsequent GNN layer may receive an output graph from a previous GNN layer and determine next layer embedded atom features for each atom based on the output graph. The one or more GNN layers may be customized Graph Isomorphism Network (GIN) layers.

The one or more GNN layers included in the embedding model may use a message-passing framework. At each of the one or more GNN layers, each node in a graph (which may be a molecule graph) may receive a message from each neighboring node. Two nodes may be neighboring nodes if the two nodes are connected by an edge in the graph. A message may be based on node features of a sending node and edge features of an edge connecting the sending node to a receiving node. For example, the one or more GNN layers may construct the message by concatenating the node features of the sending node with the edge features of the edge connecting the sending node to the receiving node.

The one or more GNN layers may use an attention mechanism to prioritize (i.e., weight) messages from neighboring nodes. An attention layer may determine a weight (which may be referred to as an edge weight) to apply to each message. The edge weight for each message may be based on node features of a node sending the message (a sending node) and node features of a node receiving the message (a receiving node). The one or more GNN layers may learn to determine the edge weight for each message based on a correlation between the node features of the sending node and the node features of the receiving node. For example, the one or more GNN layers may determine the edge weight by concatenating the node features of the sending node and the node features of the receiving node, applying a linear layer, and applying a sigmoid activation to the output. By using an attention mechanism, the embedding model may learn how to prioritize different messages sent to a receiving node based on a relationship between features of a sending node and features of the receiving node. Using the attention mechanism and edge weights that are based on features of a sending node and features of a receiving node may improve accuracy of the embedding model when used in connection with performing downstream tasks.

The following expression illustrates one example of how the one or more GNN layers may determine features xᵢ^{′} for a node i in a graph:

$${x}_{i}^{′} = {h}_{Θ} ( {x}_{i} + {\sum }_{j ∈ N ( i )} ( {x}_{j} + {e}_{j , i} ) \times {ew}_{j , i} )$$

where xᵢ^{′} is an output of a GNN layer for node i, (xⱼ+e_{j,i}) (which may be referred to as m_{j,i}) is the message from node j to node i, xⱼ is the features of node j, e_{j, i} is the features of the edge connecting node j to node i, ew_{j,i} is the edge weight for the message from node j to node i (N(i), i, and h_{θ} denotes a neural network.

The following expression illustrates one example of how ew_{j,i} may be determined:

ew_{j,i}=δ((xⱼ+xᵢ)×W_{f}+b_{f})

where ew_{j,i} is the edge weight and the attention mechanism, xⱼ is the features of the sending node, xᵢ is the features of the receiving node, W_{f} is a learned weighting coefficient, b_{f} is a learned bias coefficient, and δ is a non-linearity. W_{f} may be learned based on features of two ends of the edge.

As noted above, each of the one or more GNN layers may output embedded atom features for each atom in a molecule graph. The outputted embedded atom features may be referred to as a hidden state for the atom. An attention layer may use the hidden states (or, in a case of an attention layer associated with a first GNN layer, atom features of an input graph or updated graph) to generate edge weights for a GNN layer (which may be referred to as a next GNN layer) subsequent to a GNN layer (which may be referred to as a previous GNN layer) that generated the hidden states. The next GNN layer may receive the hidden states from the previous GNN layer as atom features and may receive the edge weights from the attention layer. The next GNN layer may output new hidden states based on the hidden states and the edge weights. The atom embedding model may include multiple attention layers and GNN layers stacked on top of each other. Each additional layer may provide visibility to further neighbors from any given node.

After generating embedded atom features using a stack of GNN layers, the atom aggregation model may generate a molecule embedding (which may also be referred to as molecule features). The atom aggregation model may generate the molecule embedding based on the embedded atom features. The atom aggregation model may first aggregate embedded atom features generated by each of the one or more GNN layers to generate aggregated atom features for each atom in the molecule graph. The atom aggregation model may then aggregate the aggregated atom features to generate the molecule features. One aggregation strategy may be based on concatenating the embedded atom features generated by each of the one or more GNN layers to generated aggregated atom features and then using an attention pooling layer to prioritize aggregated atom features of different atoms. The attention pooling layer may learn how to prioritize aggregated atom features of different atoms to calculate molecule features such that the embedding model achieves the highest accuracy in all downstream tasks.

Training using the output of a decoder to generate a back propagation gradient of the error may be used to train the embedding model. The training may result in the embedding model being sufficiently generic such that the embedding model may be used as a core in different regression, classification, or clustering models. To achieve this result the encoder-decoder model may be trained on a wide range of molecule graphs and their SMILES. By training the encoder-decoder model on a wide range of molecule graphs, the embedding model (encoder) may generate high quality molecule features and a denser embedding space that captures a wide range of important features of the molecule. Therefore, there is a higher chance that the molecule embedding contains the required information to be used in a variety of tasks. For example, such a generic embedding model may be used as a core of other models to improve accuracy and training time for the other models. Using a generic embedding model trained using the encoder-decoder architecture, as a core, may also be helpful when there are insufficient labeled data for the downstream prediction task. The embedding space itself may also be used to find similar molecules or find molecule clusters that share interesting properties (such as solubility).

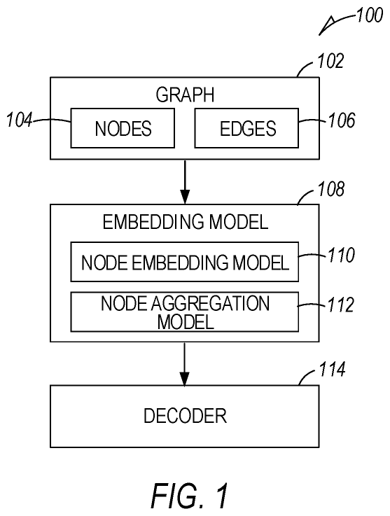

FIG. 1 illustrates an encoder-decoder system 100. The system 100 may include a graph 102, an embedding model 108, and a decoder 114.

The graph 102 may be a data structure. The graph 102 may contain information regarding real-world entities and relationships between the real-world entities. As one example, the graph 102 may represent a molecule and contain information regarding atoms that form the molecule and regarding bonds between and among the atoms of the molecule. In the case of a molecule, the graph 102 may be based in part on a SMILES of the molecule. As another example, the graph 102 may represent a social network, a biological system, or a financial system.

The graph 102 may include nodes 104 (which may also be referred to as vertices) and edges 106 (which may also be referred to as links).

The nodes 104 may represent component entities that make up the graph 102. The nodes 104 may have features. The features may contain information regarding properties of the nodes 104. For example, consider that the graph 102 represents a molecule and the nodes 104 represent atoms within the molecule. The atoms within the molecule may have certain properties such as atomic numbers and chirality. The features of the nodes 104 may include the properties of the atoms. The features of the nodes 104 may be based on the properties of the atoms. For example, the features of the nodes 104 may be determined using one-hot encoding and/or linear mapping based on the properties of the atoms. The features of the nodes 104 may be represented in a vector.

The edges 106 may represent relationships between pairs of nodes. The edges 106 may be directional or non-directional. The edges 106 may have features that contain information regarding the relationships between the pairs of nodes. For example, in the situation in which the graph 102 represents a molecule, the edges 106 may represent bonds between atoms within the molecule. The bonds between the atoms within the molecule may have certain properties, such as bond type and bond direction. The features of the edges 106 may include the properties of the bonds. The features of the edges 106 may be based on the properties of the edges 106. For example, the features of the edges 106 may be generated based on the properties of the bonds. The features of the edges 106 may be represented in a vector.

The embedding model 108 may include a machine learning model that receives a graph (such as the graph 102) and outputs a representation of the graph in an embedding space. The embedding space may be a Euclidean space. The embedding space may be any space in which a point in an n-dimensional embedding space can be defined using a vector of size n. The embedding space may have a defined number of dimensions. Each point in the embedding space may be defined by certain values for each dimension. The representation of the graph in the embedding space may be a vector having a same number of dimensions as the embedding space. The embedding space may be denser than a space in which the graph exists. For example, the graph may represent a molecule. The molecule may exist in a space of all molecules. The embedding model 108 may output a representation of the molecule in an embedding space. The representation of the molecule in the embedding space may be molecule features of the molecule. The embedding space may be denser than the space of all molecules.

The embedding model 108 may include a node embedding model 110 and a node aggregation model 112.

The node embedding model 110 may include one or more GNN layers. Each of the one or more GNN layers may receive an input graph and output an embedded graph (which may be a hidden state). At each of the one or more GNN layers, each node in the input graph may have a corresponding node in the embedded graph. Each node in the input graph may have input features. Each corresponding node in the embedded graph may have embedded features. Embedded features of an output node in an embedded graph (which may correspond to an input node in an input graph) may contain more information about the output node than is contained in input features of the input node. Each of the one or more GNN layers may learn to take the input features (which may have no correlation or an unknown correlation) and neighborhood information and map the input features and the neighborhood information to a singular representation (embedded features) that has all that information embedded into it. The one or more GNN layers may learn to determine the embedded features to achieve a highest accuracy on all downstream tasks. Each of the one or more GNN layers may access structure information contained in the input graph in determining the embedding features.

At least one of the one or more GNN layers may use a message-passing framework and an attention mechanism to determine, based on an input graph, embedded features for an embedded graph. Each node in the input graph may receive a message from each neighboring node in the input graph. A neighboring node of a node may be any node connected to the node by an edge. A message from a neighboring node to a receiving node may be based on features of the neighboring node and features of an edge connecting the neighboring node to the receiving node. A GNN layer may use messages received by a receiving node from neighboring nodes to determine embedded features of the receiving node.

A GNN layer may use the attention mechanism to weight each of the messages received by the receiving node in determining the embedded features. The GNN layer may receive weights for each of the messages from an attention layer. The attention layer may, for each message, determine a weight based on features of a node in the input graph that is sending the message and features of a node in the input graph that is receiving the message. The weights may communicate to the GNN layer which neighboring node's information is most important. The attention layer may learn how to put weights on the messages. The attention layer may learn how to put weights on the messages based on a correlation of features of a receiving node and features of a sending node. Utilizing weights determined based on features of a receiving node and features of a sending node, may increase in order to determine embedded features may increase an accuracy of the embedding model 108 in connection with performing downstream tasks. These weights may also be used to investigate and identify portions of a molecule structure that were more important during the inference.

The node aggregation model 112 may determine molecule features for an input graph (such as the graph 102) based on embedded graphs generated by the one or more GNN layers. The molecule features may define a point in an embedding space of the input graph. The node aggregation model 112 may determine aggregated node features for each node in the input graph. The aggregated node features for a node may be based on embedded features of the node in the embedded graphs. For example, the node aggregation model 112 may determine the aggregated node features by determining an average of the embedded features of the node in the embedded graphs.

The node aggregation model 112 may determine the molecule features based on the aggregated node features of the nodes. The node aggregation model 112 may prioritize aggregated node features of some nodes of the input graph over other nodes of the input graph. The node aggregation model 112 may determine a weight to apply to aggregated node features of each node in the input graph in determining the molecule features. The node aggregation model 112 may learn to determine weights to apply to aggregated node features to achieve a highest accuracy on downstream tasks.

A decoder 114 may receive an output of the embedding model 108. The output of the embedding model 108 may be a vector representative of the molecule features. The generated vector from the molecule embedding model is the encoder output that is used in the decoder to predict the SMILES sequence. The decoder in one embodiment is an attention based RNN (recurrent neural network), where in each iteration the next token in the smiles sequence is predicted based on the next value in the encoder output, the attention output, and the previous hidden state of the RNN. When the RNN predicts the end token, that will be the end of that SMILES sequence. A loss function is used during training to calculate the gradient that should be propagated through the network which contains the embedding model 108.

Attention allows the decoder network to focus on a different part of the encoder's outputs for every step of the decoder's outputs. First, we calculate a set of attention weights. These will be multiplied by the encoder output vectors to create a weighted combination. The result should contain information about that specific part of the input sequence, and thus help the decoder choose the right output SMILES token.

The loss function in this model is based on the cross entropy of the decoder output and the tokenized SMILES at any given point of the SMILES sequence.

The output of the embedding model 108 may be used to map the input graph to a point in the embedding space. The embedding space may allow for determining a distance between the input graph and other molecules mapped to the embedding space. Molecules that are within a threshold distance in the embedding space may have similar properties.

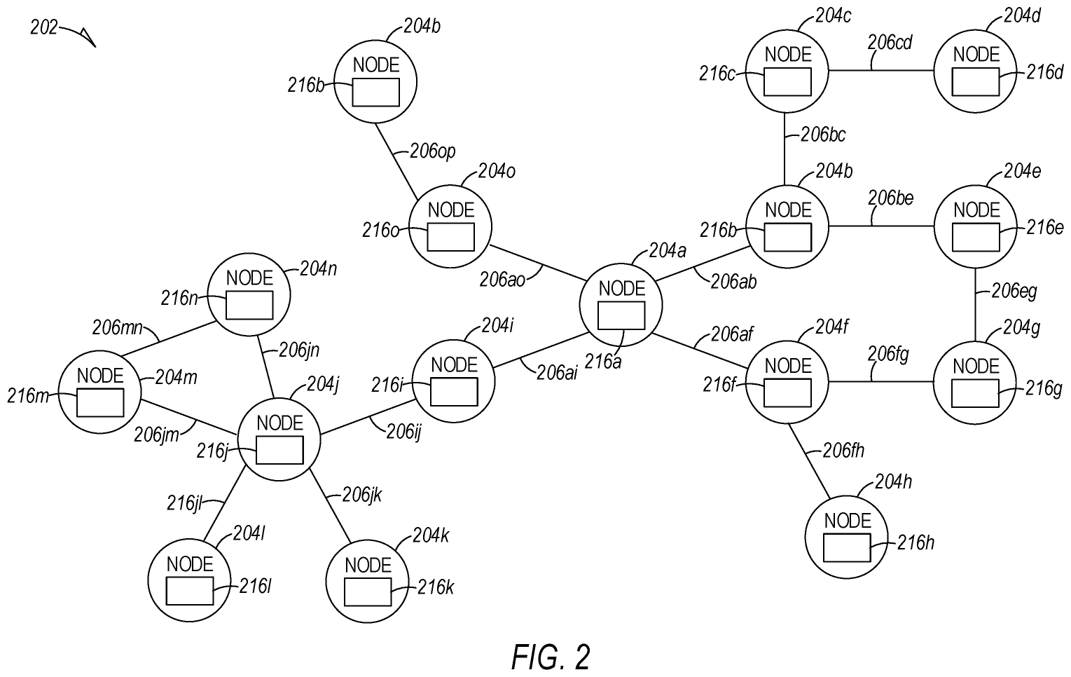

FIG. 2 illustrates an example graph 202. The graph 202 may represent a molecule. The graph 202 may be an input to an embedding model (such as the embedding model 108), an input to an embedding layer, an output of an embedding layer, a hidden state within an embedding model, or an output of a node embedding model (such as the node embedding model 110).

The graph 202 may include nodes 204a, 204b, 204c, 204d, 204e, 204f, 204g, 204h, 204i, 204j, 204k, 204l, 204m, 204n, 204o, and 204p, referred to as 204a-p. In other designs, the graph 202 may include fewer or more nodes. Each of the nodes 204a-p may represent an atom in a molecule. The nodes 204a-p may include features 216a, 216b, 216c, 216d, 216e, 216f, 216g, 216h, 216i, 216j, 216k, 216l, 216m, 216n, 216o, and 216p, referred to as 216a-p. The features 216a-p may be based on properties of atoms represented by the nodes 204a-p. For example, the node 204a may represent a first atom in a molecule. The first atom may have an atomic number, a chirality, and a charge. The features 216a may be based on the atomic number, the chirality, and the charge of the first atom. The features 216a-p may be represented in vectors. The features 216a-p may be embedded features.

The graph 202 may include edges 206ab, 206bc, 206be, 206cd, 206eg, 206af, 206fg, 206fh, 206ai, 206ij, 206jk, 206jl, 206jm, 206jn, 206mn, 206ao, 206op (which may be referred to as edges 206ab-op). The edges 206ab-op may represent bonds in the molecule. Each of the edges 206ab-op may include edge features. The edge features may be based on properties of the bonds represented by the edges 206ab-op. For example, the edge 206ab may represent a first bond in a molecule. The first bond may have a bond type and a bond direction. Edge features of the edge 206ab may be based on the bond type and the bond direction. The edge features may be represented in vectors.

In situations in which the graph 202 is a hidden state within an embedding model, the features 216a-p may be based on more than properties of the atoms that the nodes 204a-p represent. Consider an example in which the graph 202 is a hidden state (an output) of a first graph neural network layer in an embedding model. Assume that the first graph neural network layer receives an input graph. The features 216a of the node 204a may be based not only on properties of an atom that the node 204a represents but may also be based on features of neighboring nodes (which, if temporarily viewing the graph 202 as the input graph, would be the features 216b of the node 204b, the features 216f of the node 204f, the features 216i of the node 204i, and the features 216o of the node 204o). The features 216a of the node 204a may further be based on edge properties of edges that connect the node 204a to its neighboring nodes (which, if temporarily viewing the graph 202 as the input graph, would be the edge 206ab, the edge 206af, the edge 206ai, and the edge 206ao). In a situation in which the first graph neural network layer utilizes an attention mechanism, the features 216a may be based on edge weights. The edge weights may be based on features of the neighboring nodes of the node 204a in the input graph and the features 216a in the input graph.

Consider another example in which the graph 202 is a hidden state (an output) of a second graph neural network layer that is subsequent to the first graph neural network layer of the example above. In such an example, the features 216a of the node 204a may be further based not only on features of neighboring nodes of the node 204a but also on features of nodes that neighbor the neighboring nodes of the node 204a (which, if temporarily viewing the graph 202 as an output from the first graph neural network layer, would be the features 216c of the node 204c, the features 216e of the node 204e, the features 216g of the node 204g, the features 216h of the node 204h, the features 216j of the node 204j, and the features 216p of the node 204p). The features 216a of the node 204a may further be based on edge features (which, if temporarily viewing the graph 202 as the output from the first graph neural network layer, would be the edge 206bc, the edge 206be, the edge 206fg, the edge 206fh, the edge 206ij, and the edge 206op). In a situation in which the second graph neural network layer utilizes an attention mechanism, the features 216a may be based on edge weights. The edge weights may be based on features of the neighboring nodes of the node 204a in the output from the first graph neural network layer and the features 216a in the output from the first graph neural network layer.

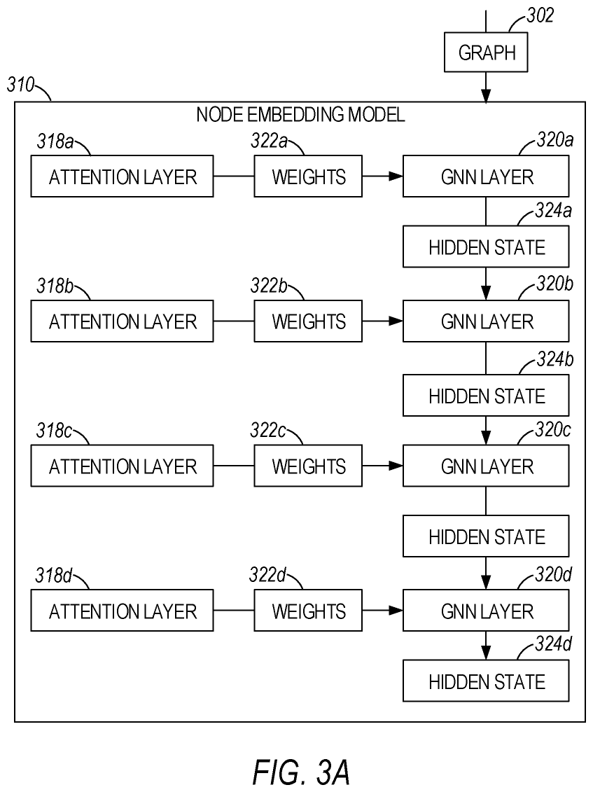

FIG. 3A illustrates a node embedding model 310. The node embedding model 310 may receive a graph 302. The graph 302 may represent a molecule. The graph 302 may be the graph 102 or the graph 202.

The node embedding model 310 may include attention layers 318a-d and GNN layers 320a-d. The GNN layers 320a-d may determine hidden states 324a-d, and the attention layers 318a-d may determine weights 322a-d. Although the node embedding model 310 includes four GNN layers, in other designs, a node embedding model may include fewer GNN layers (such as a single GNN layer) or more GNN layers. Although the node embedding model 310 includes an attention layer for each GNN layer, in other designs, one or more GNN layers may not have an associated attention layer. For example, a node embedding model may include a first GNN layer and a second GNN layer. The first GNN layer may not have an associated attention layer while the second GNN layer may have an associated attention layer.

The GNN layer 320a may receive an input graph. The input graph may be the graph 302 or a modified version of the graph 302. For example, the node embedding model 310 may use a mapping layer to map atomic numbers to a dense feature space and replace the atomic number in each node with generated features. Each node in the input graph may receive a message from each neighboring node. A node that receives a message may be referred to as a receiving node and a node that sends the message may be referred to as a sending node. The message may include features of the sending node and features of an edge connecting the sending node and the receiving node. The features of the edge connecting the sending node and the receiving node may be different from features of an edge connecting the receiving node to the sending node. In other words, edges of the input graph may be directional.

The attention layer 318a may receive the graph 302 or a modified version of the graph (or a subset of the foregoing). The attention layer 318a may output the weights 322a to the GNN layer 320a. The weights 322a may include a weight for each message sent by a sending node to a receiving node. The attention layer 318a may determine the weights 322a based on features of the sending node and features of the receiving node. For example, the attention layer 318a may determine the weights 322a based in part on concatenating the features of the sending node and the features of the receiving node. The attention layer 318a may learn how to determine the weights 322a based on a relationship between features of a sending node and features of a receiving node. For example, the attention layer 318a may learn a weighting coefficient and a bias coefficient for determining the weights 322a. The attention layer 318a may apply the weighting coefficient to a concatenation of the features of the sending node and the features of the receiving node. The attention layer 318a may concatenate the bias coefficient to a result of the foregoing calculation. The attention layer 318a may then apply a sigmoid.

The GNN layer 320a may determine the hidden state 324a for the input graph. The hidden state 324a may be a graph identical to the input graph in terms of its structure, except that nodes of the hidden state 324a may have features different from input features of nodes in the input graph. The features of a node of the hidden state 324a may be referred to as embedded features of the node or a hidden state of the node. The GNN layer 320a may determine embedded features for each node in the hidden state 324a. The embedded features for each node in the hidden state 324a may be based on messages received by the node, weights associated with the messages received by the node (which may be contained in the weights 322a), and input features of the node in the input graph. The GNN layer 320a may learn how to determine the embedded features for each node in the hidden state 324a such that one or more downstream tasks may be performed with a lowest error. Edges of the hidden state 324a may have edge features identical to edges of the input graph.

The GNN layer 320b may receive the hidden state 324a. Each node in the hidden state 324a may receive a message from each neighboring node. The message may include features of the sending node and features of an edge connecting the sending node and the receiving node. The features of the sending node may give the receiving node visibility to features of nodes that neighbor the sending node.

The attention layer 318b may receive the hidden state 324a or a subset of the hidden state 324a. The attention layer 318b may output the weights 322b to the GNN layer 320b. The weights 322b may include a weight for each message sent by a sending node to a receiving node. The attention layer 318b may determine the weights 322b based on features of the sending node and features of the receiving node. For example, the attention layer 318b may determine the weights 322b based in part on concatenating the features of the sending node and the features of the receiving node. The attention layer 318b may learn how to determine the weights 322b based on a relationship between features of a sending node and features of a receiving node.

The GNN layer 320b may determine the hidden state 324b for the hidden state 324a. The hidden state 324b may be a graph identical to the hidden state 324a structurally, except that nodes of the hidden state 324b may have features different from features of nodes of the hidden state 324a. The features of a node of the hidden state 324b may be referred to as embedded features of the node or a hidden state of the node. The GNN layer 320b may determine the embedded features for each node in the hidden state 324b. The embedded features for each node in the hidden state 324b may be based on messages received by the node, weights associated with the messages received by the node (which may be contained in the weights 322b), and features of the node in the hidden state 324a. The GNN layer 320b may learn how to determine the embedded features for each node in the hidden state 32ba such that one or more downstream tasks may be predicted with the lowest error. Edges of the hidden state 324b may have edge features identical to edges of the hidden state 324a.

The GNN layer 320c may receive the hidden state 324b. Each node in the hidden state 324b may receive a message from each neighboring node. The message may include features of the sending node and features of an edge connecting the sending node and the receiving node. The features of the sending node may give the receiving node visibility to features of nodes that neighbor neighbors of the sending node (two hops information).

The attention layer 318c may receive the hidden state 324b or a subset of the hidden state 324b. The attention layer 318c may output the weights 322c to the GNN layer 320c. The weights 322c may include a weight for each message sent by a sending node to a receiving node. The attention layer 318c may determine the weights 322c based on features of the sending node and the receiving node. For example, the attention layer 318c may determine the weights 322c based on concatenating the features of the sending node and the features of the receiving node. The attention layer 318c may learn how to determine the weights 322c based on a relationship between features of a sending node and features of a receiving node. The attention layer 318c may learn how to determine the weights 322c in a same way as the attention layer 318a may learn to determine the weights 322a.

The GNN layer 320c may determine the hidden state 324c for the hidden state 324b. The hidden state 324c may be a graph identical to the hidden state 324b structurally, except that nodes of the hidden state 324c may have features different from features of nodes of the hidden state 324b. The features of a node of the hidden state 324c may be referred to as embedded features of the node or a hidden state of the node. The GNN layer 320c may determine the embedded features for each node in the hidden state 324c. The embedded features for each node in the hidden state 324c may be based on messages received by the node, weights associated with the messages received by the node (which may be contained in the weights 322c), and features of the node in the hidden state 324b. The GNN layer 320c may learn how to determine the embedded features for each node in the hidden state 324c such that one or more downstream tasks may be predicted with the lowest error. Edges of the hidden state 324c may have edge features identical to edges of the hidden state 324b.

The GNN layer 320d may receive the hidden state 324c. Each node in the hidden state 324c may receive a message from each neighboring node. The message may include features of the sending node and features of an edge connecting the sending node and the receiving node. The features of the sending node may give the receiving node visibility to features of nodes that neighbor neighbors of neighbors of the sending node (three hops visibility).

The attention layer 318d may receive the hidden state 324c or a subset of the hidden state 324c. The attention layer 318d may output the weights 322d to the GNN layer 320d. The weights 322d may include a weight for each message sent by a sending node to a receiving node. The attention layer 318d may determine the weights 322d based on features of the sending node and features of the receiving node. For example, the attention layer 318d may determine the weights 322d based on concatenating the features of the sending node and the features of the receiving node. The attention layer 318d may learn how to determine the weights 322d based on a relationship between features of a sending node and features of a receiving node.

The GNN layer 320d may determine a hidden state 324d for the hidden state 324c. The hidden state 324d may be a graph identical to the hidden state 324c except that nodes of the hidden state 324d may have features different from features of nodes of the hidden state 324c. The features of a node of the hidden state 324d may be referred to as embedded features of the node or a hidden state of the node. The GNN layer 320d may determine the embedded features for each node in the hidden state 324d. The embedded features for each node in the hidden state 324d may be based on messages received by the node, weights associated with the messages received by the node (which may be contained in the weights 322d), and features of the node in the hidden state 324c. The GNN layer 320d may learn how to determine the embedded features for each node in the hidden state 324d such that one or more downstream tasks may be predicted with the lowest error. Edges of the hidden state 324d may have edge features identical to edges of the hidden state 324c.

The embedded features for nodes included in the hidden states 324a-d may have a same size or different sizes.

FIG. 3B illustrates a receiving node and four sending nodes that may exist in the graph 302, a graph input into the GNN layer 320a, or the hidden states 304a-c.

A node 304a may include features 316a.

The node 304a may receive a message 334ba from node 304b. The node 304b may include features 316b. Edge 306ba may include features 332-1. The message 334ba may be based on the features 316b and the features 332-1.

The node 304a may receive a message 334ca from node 304c. The node 304c may include features 316c. Edge 306ca may include features 332-2. The message 334ca may be based on the features 316c and the features 332-2.

The node 304a may receive a message 334da from node 304d. The node 304d may include features 316d. Edge 306da may include features 332-3. The message 334da may be based on the features 316d and the features 332-3.

The node 304a may receive a message 334ea from node 304e. The node 304e may include features 316e. Edge 306ea may include features 332-4. The message 334ea may be based on the features 316e and the features 332-4.

Assume the node 304a receives the messages 334ba, 334ca, 334da, 334ea within the GNN layer 320b shown in FIG. 3A. The node 304a may apply a weight to each of the messages 334ba, 334ca, 334da, 334ea. The node 304a may apply a weight to each of the messages 334ba, 334ca, 334da, 334ea based on the weights 322b. The weights 322b may include a weight for each of the messages 334ba, 334ca, 334da, 334ea. For example, the weights 322b may include a first weight for the message 334ba, a second weight for the message 334ca, a third weight for the message 334da, and a fourth weight for the message 334ea.

The attention layer 318b may determine the weights 322b. The attention layer 318b may determine the first weight for the message 334ba based on the features 316b and the features 316a. The attention layer 318b may determine the second weight for the message 334ca based on the features 316c and the features 316a. The attention layer 318b may determine the third weight for the message 334da based on the features 316d and the features 316a. The attention layer 318b may determine the fourth weight for the message 334ea based on the features 316e and the features 316a. The first weight, the second weight, the third weight, and the fourth weight may be further based on a weighting coefficient and a bias coefficient. The attention layer 318b may learn the weighting coefficient and the bias coefficient.

Continuing with this example, the GNN layer 320b may determine embedded features for the node 304a based on the messages 334ba, 334ca, 334da, 334ea, the first weight, the second weight, the third weight, the fourth weight, and the features 316a. For example, the message 334ba may be a concatenation of the features 332-1 and the features 316b. The message 334ca may be a concatenation of the features 332-2 and the features 316c. The message 334da may be a concatenation of the features 332-3 and the features 316d. The message 334ea may be a concatenation of the features 332-4 and the features 316e. The GNN layer 320b may apply the first weight to the message 334ba to generate a weighted first message. The GNN layer 320b may apply the second weight to the message 334ca to generate a weighted second message. The GNN layer 320b may apply the third weight to the message 334da to generate a weighted third message. The GNN layer 320b may apply the fourth weight to the message 334ea to generate a weighted fourth message. The GNN layer 320b may sum the weighted first message, the weighted second message, the weighted third message, and the weighted fourth message to generate a message sum. The GNN layer 320b may concatenate the message sum and the features 316a to generate intermediate features. The GNN layer 320b may determine the hidden state for the node 304a based on the intermediate features. The GNN layer 320b may learn to determine the hidden state for the node 304a based on the intermediate features in order to achieve the lowest error on one or more downstream tasks. Utilizing the first weight, the second weight, the third weight, and the fourth weight may improve the ability of the GNN layer 320b to capture different properties of a molecule (and an embedding model that includes the GNN layer 320b). These weights may also make the node embedding model 310 more transparent and explainable because the weights may make it possible to see which part of a molecule structure played a more important role during the inference.

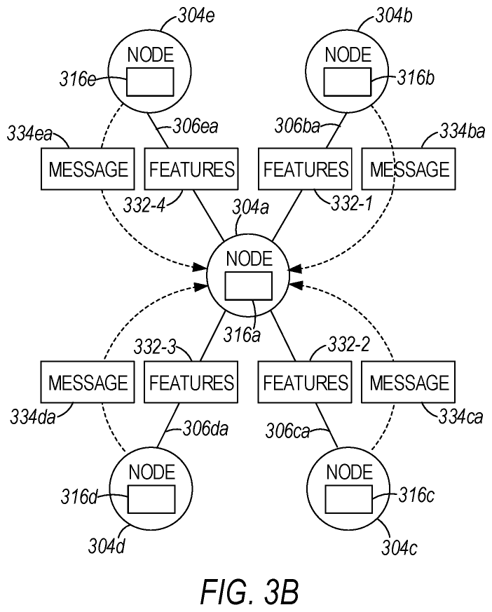

FIG. 4 illustrates a node aggregation model 412. The node aggregation model 412 may include node aggregation 428, graph aggregation 430, and an attention pooling layer 426.

Node aggregation 428 may aggregate embedded features of each node in a graph to generate aggregated node features for each node in the graph. The aggregated node features for each node in the graph may represent aggregated atom features when the graph represents a molecule. Consider the node embedding model 310. The node aggregation 428 may, for each node in the graph 302, aggregate embedded features for the node contained in the hidden states 324a-d to generate aggregated node features for the graph 302. The node aggregation 428 may apply any of a variety of aggregation policies possible for set-to-one mapping in order to determine the aggregated node features.

Consider a first node in the graph has first embedded features in the hidden state 324a, second embedded features in the hidden state 324b, third embedded features in the hidden state 324c, and fourth embedded features in the hidden state 324d. One aggregation policy may involve the node aggregation 428 concatenating the first embedded features, the second embedded features, the third embedded features, and the fourth embedded features to determine aggregated node features (which may also be referred to as final node features) for the node. As another example, the node aggregation 428 may select embedded features contained in one of the hidden states 324a-d (such as the fourth embedded features for the node in the hidden state 324d) as the final node features for the node. As another example, the node aggregation 428 may calculate a mean or a sum of the first embedded features, the second embedded features, the third embedded features, and the fourth embedded features.

As another example, the node aggregation 428 may determine a maximum value of each axis in the first embedded features, the second embedded features, the third embedded features, and the fourth embedded features. Assume that the first embedded features, the second embedded features, the third embedded features, and the fourth embedded features are each vector having n dimensions. For each dimension in the first embedded features, the second embedded features, the third embedded features, and the fourth embedded features, the node aggregation 428 may choose a maximum value among the first embedded features, the second embedded features, the third embedded features, and the fourth embedded features. The maximum value for each dimension is used to form the aggregated node features of the node.

Graph aggregation 430 may aggregate the aggregated node features determined by the node aggregation 428 to determine graph features for a graph. The graph features may be molecule features when the graph represents a molecule. The graph features may define a location of the graph in an embedding space. The graph aggregation 430 may apply any of a variety of aggregation policies to determine the graph features. For example, the graph aggregation 430 may apply any of the policies described above with respect to aggregating embedded features for a node.

The graph aggregation 430 may utilize an attention pooling layer 426 to determine the graph features. The attention pooling layer 426 may learn how to weight aggregated node features of nodes in a graph such that the graph aggregation 430 determines graph features that allow an embedding model to achieve the lowest error in downstream tasks. For example, consider a graph that includes a first node and a second node. Assume the first node has first aggregated node features and the second node has second aggregated node features. The attention pooling layer 426 may determine a first weight to apply to the first aggregated node features and a second weight to apply to the second aggregated node features. The first weight may be different from the second weight.

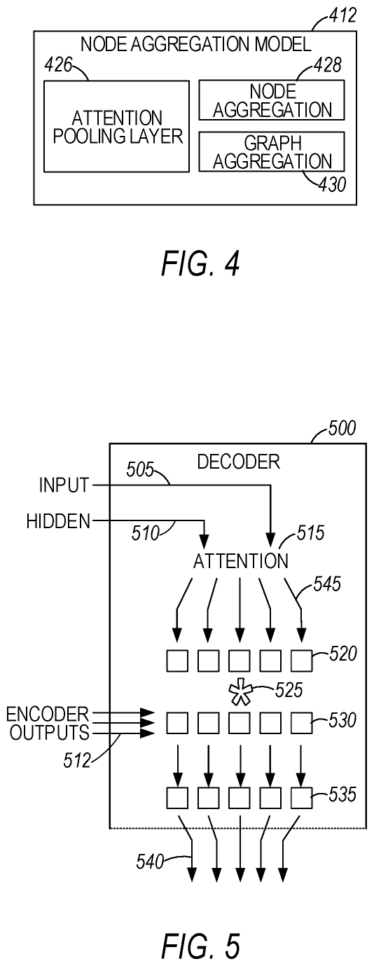

FIG. 5 illustrates a decoder 500 that is used to produce a text based representation of a molecule from the output vector of the encoder. The vector encodes contextual and relational information from the molecule graph. The output vector from the encoder is used as an input context to the decoder.

At every step of decoding, the decoder 500 is given an input token 505 and a hidden state 510 and encoder output 512 as the context. The initial input token 505 may be a start-of-string <SOS> (which represents a start of the sentence), and the first hidden state 510 is initialized to a zero tensor. For the subsequent steps, the previous decoder hidden state will be used as hidden state 510 and the next value in the encoder output will he used as input 505.

An attention 515 layer allows the decoder network to “focus” on a different part of the encoder's outputs for every step of the decoder's own outputs. A set of attention weights 520 is first calculated. The attention weights 520 will be multiplied at 525 by the encoder output vectors 530 to create a weighted combination 535. The weighted combination 535 contains information about that specific part of the input, and thus help the decoder 500 choose the right output words 540 on every step of decoding (each decoded token).

Calculating the attention weights 520 may done with a feed-forward layer 545, using the decoder's input 505 and hidden state 510 as inputs. Because there are molecule graphs of all sizes in the training data, to train the feed-forward layer, all the tokenized SMILES (character based representation of the molecule) are padded to a specific length. The output of the decoder should be the same length as well.

The decoder 500 also identifies a gradient of the error between the decoded output and a label for the molecule via a loss function for backpropagation during training of the encoder-decoder model. The decoder 500 is an attention based RNN (recurrent neural network) in one embodiment, where each iteration predicts a next token 540 in a SMILES sequence based on the next value in the encoder output, the attention output, and the previous hidden state of the RNN. When the RNN predict the end token, that will be the end of the predicted SMILES sequence.

The loss function in one embodiment is based on the cross entropy of the decoder 500 output and the tokenized SMILES at any given point of the SMILES sequence. In further embodiments a Connectionist temporal classification function (CTC Loss) may be used as the loss function. The encoder-decoder architecture 100 provides a way to train the molecule encoder without labeled data (unsupervised training).

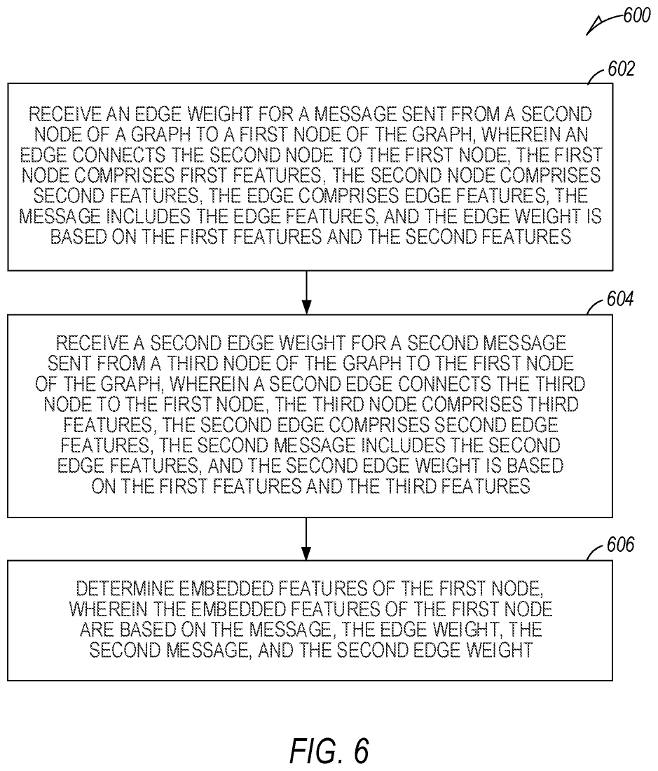

FIG. 6 illustrates an example computer implemented method 600 of determining embedded features of the encoder model. Method 600 may include receiving at operation 602 an edge weight for a message sent from a second node of a graph to a first node of the graph, wherein an edge connects the second node to the first node, the first node comprises first features, the second node comprises second features, the edge comprises edge features, the message includes the edge features, and the edge weight is based on the first features and the second features. The edge weight may be further based on a learned weighting coefficient. The graph may represent a molecule. The graph may be based on a SMILES of the molecule. A graph neural network may receive the edge weight. The graph neural network may be a graph isomorphism network.

The method 600 may include receiving at operation 604 a second edge weight for a second message sent from a third node of the graph to the first node of the graph, wherein a second edge connects the third node to the first node, the third node comprises third features, the second edge comprises second edge features, the second message includes the second edge features, and the second edge weight is based on the first features and the third features. The graph neural network may receive the second edge weight. The second edge weight may be further based on the learned weighting coefficient.

The method 600 may include determining at operation 606 embedded features of the first node, wherein the embedded features of the first node are based on the message, the edge weight, the second message, and the second edge weight. The graph neural network may determine the embedded features of the first node.

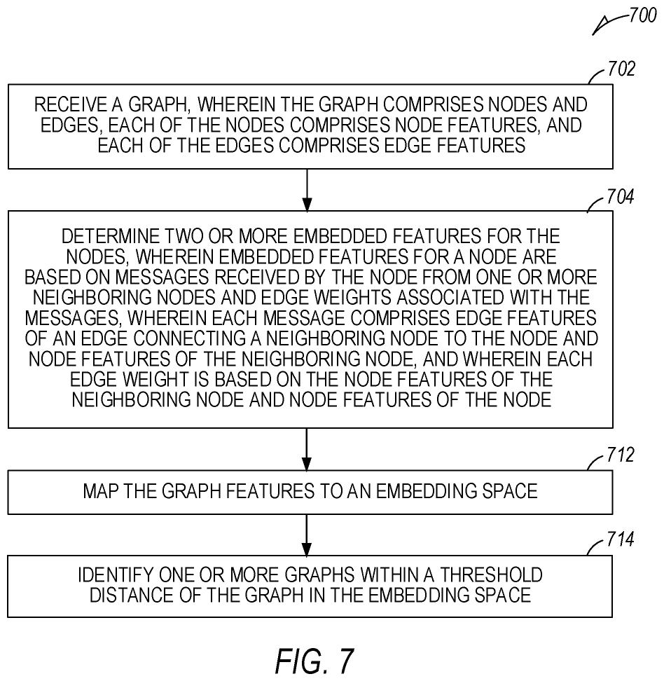

FIG. 7 illustrates an example method 700 of receiving a graph of a molecule and identifying one or more graphs within a threshold difference in an molecule embedding space.

Method 700 may include receiving at operation 702 a graph, wherein the graph comprises nodes and edges, each of the nodes comprises node features, and each of the edges comprises edge features. The graph may represent a molecule. The graph may be based on a simplified molecular-input line-entry system (SMILES) of the molecule.

The method 700 may include determining at operation 704 two or more embedded features for the nodes as described with respect to FIG. 6, wherein embedded features for a node are based on messages received by the node from one or more neighboring nodes and edge weights associated with the messages, wherein each message comprises edge features of an edge connecting a neighboring node to the node and node features of the neighboring node, and wherein each edge weight is based on the node features of the neighboring node and node features of the node. Two or more graph neural network layers may determine the two or more embedded features for the nodes.

The method may include mapping at operation 712 the graph features to an embedding space.

The method may include identifying at operation 714 one or more graphs within a threshold distance of the graph in the embedding space.

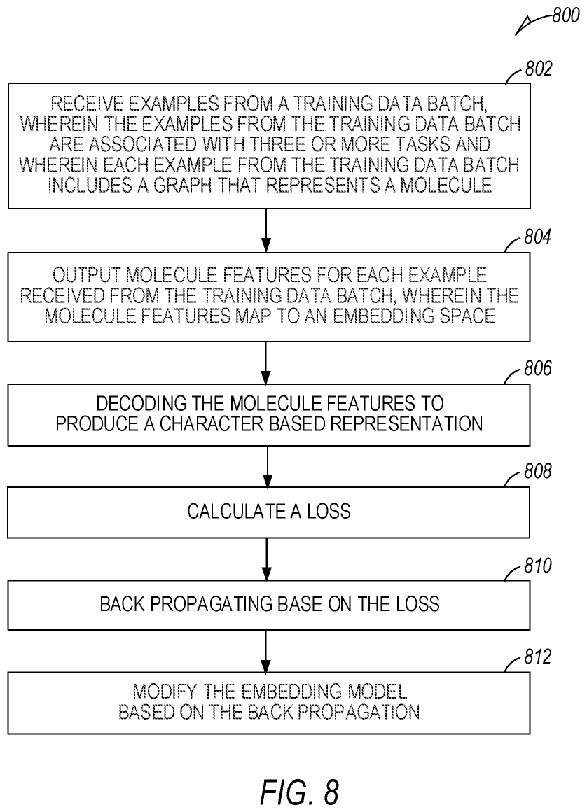

FIG. 8 illustrates an example computer implemented method 800.

The method 800 may include a receiving operation 802 to receive examples from a training data batch, wherein the examples from the training data batch comprises molecule graphs that represent various molecules. An embedding model may receive the examples. The embedding model may include one or more graph neural network layers and one or more attention layers. The graph may include nodes and edges. The one or more graph neural network layers may use a message-passing framework. The one or more attention layers may determine edge weights to be applied to messages received by a receiving node in the graph from one or more sending nodes in the graph based on how the message-passing framework propagates information in the graph. The edge weights may be based on features of the receiving node and the one or more sending nodes and on a weighting coefficient.

The method 800 may include an outputting operation 804 molecule features for each example received from the training data batch, wherein the molecule features map to an embedding space. The embedding model may output the molecule features. The molecule features may be based in part on the edge weights and the messages.

The method 800 may include a decoding operation 806 the molecule features to predict a character-based representation. An attention based recurrent neural network decoder may be used in one embodiment. At operation 808, a loss function may be applied to the output of the decoder and a character-based representation of the input molecule graph to calculate a loss. At operation 810, the loss is backpropagated through the encoder-decoder model. Learnable weights of the embedding model (encoder) are changed based on the back propagation at operation 812. The one or more attention layers may learn how to prioritize different messages based on the back propagation.

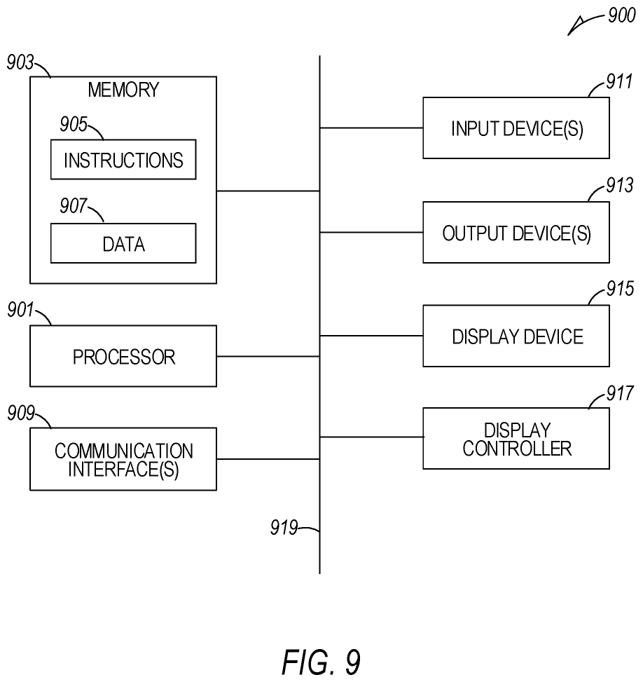

Reference is now made to FIG. 9. One or more computing devices 900 can be used to implement at least some aspects of the techniques disclosed herein. FIG. 9 illustrates certain components that can be included within a computing device 900.

The computing device 900 includes a processor 901 and memory 903 in electronic communication with the processor 901. Instructions 905 and data 907 can be stored in the memory 903. The instructions 905 can be executable by the processor 901 to implement some or all of the methods, steps, operations, actions, or other functionality that is disclosed herein. Executing the instructions 905 can involve the use of the data 907 that is stored in the memory 903. Unless otherwise specified, any of the various examples of modules and components described herein can be implemented, partially or wholly, as instructions 905 stored in memory 903 and executed by the processor 901. Any of the various examples of data described herein can be among the data 907 that is stored in memory 903 and used during execution of the instructions 905 by the processor 901.

Although just a single processor 901 is shown in the computing device 900 of FIG. 9, in an alternative configuration, a combination of processors (e.g., an Advanced RISC (Reduced Instruction Set Computer) Machine (ARM) and a digital signal processor (DSP)) could be used.

The computing device 900 can also include one or more communication interfaces 909 for communicating with other electronic devices. The communication interface(s) 909 can be based on wired communication technology, wireless communication technology, or both. Some examples of communication interfaces 909 include a Universal Serial Bus (USB), an Ethernet adapter, a wireless adapter that operates in accordance with an Institute of Electrical and Electronics Engineers (IEEE) 802.11 wireless communication protocol, a Bluetooth® wireless communication adapter, and an infrared (IR) communication port.

The computing device 900 can also include one or more input devices 911 and one or more output devices 913. Some examples of input devices 911 include a keyboard, mouse, microphone, remote control device, button, joystick, trackball, touchpad, and lightpen. One specific type of output device 913 that is typically included in a computing device 900 is a display device 915. Display devices 915 used with embodiments disclosed herein can utilize any suitable image projection technology, such as liquid crystal display (LCD), light-emitting diode (LED), gas plasma, electroluminescence, wearable display, or the like. A display controller 917 can also be provided, for converting data 907 stored in the memory 903 into text, graphics, and/or moving images (as appropriate) shown on the display device 915. The computing device 900 can also include other types of output devices 913, such as a speaker, a printer, etc.

The various components of the computing device 900 can be coupled together by one or more buses, which can include a power bus, a control signal bus, a status signal bus, a data bus, etc. For the sake of clarity, the various buses are illustrated in FIG. 9 as a bus system 919.

The techniques disclosed herein can be implemented in hardware, software, firmware, or any combination thereof, unless specifically described as being implemented in a specific manner. Any features described as modules, components, or the like can also be implemented together in an integrated logic device or separately as discrete but interoperable logic devices. If implemented in software, the techniques can be realized at least in part by a non-transitory computer-readable medium having computer-executable instructions stored thereon that, when executed by at least one processor, perform some or all of the steps, operations, actions, or other functionality disclosed herein. The instructions can be organized into routines, programs, objects, components, data structures, etc., which can perform particular tasks and/or implement particular data types, and which can be combined or distributed as desired in various embodiments.

The term “processor” can refer to a general purpose single- or multi-chip microprocessor (e.g., an Advanced RISC (Reduced Instruction Set Computer) Machine (ARM)), a special purpose microprocessor (e.g., a digital signal processor (DSP)), a microcontroller, a programmable gate array, or the like. A processor can be a central processing unit (CPU). In some embodiments, a combination of processors (e.g., an ARM and DSP) could be used to implement some or all of the techniques disclosed herein.

The term “memory” can refer to any electronic component capable of storing electronic information. For example, memory may be embodied as random access memory (RAM), read-only memory (ROM), magnetic disk storage media, optical storage media, flash memory devices in RAM, various types of storage class memory, on-board memory included with a processor, erasable programmable read-only memory (EPROM), electrically erasable programmable read-only memory (EEPROM) memory, registers, and so forth, including combinations thereof.

The terms computer-readable medium, machine readable medium, and storage device do not include carrier waves or signals to the extent carrier waves and signals are deemed too transitory.

The steps, operations, and/or actions of the methods described herein may be interchanged with one another without departing from the scope of the claims. In other words, unless a specific order of steps, operations, and/or actions is required for proper functioning of the method that is being described, the order and/or use of specific steps, operations, and/or actions may be modified without departing from the scope of the claims.

The term “determining” (and grammatical variants thereof) can encompass a wide variety of actions. For example, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The terms “comprising,” “including,” and “having” are intended to be inclusive and mean that there can be additional elements other than the listed elements. Additionally, it should be understood that references to “one embodiment” or “an embodiment” of the present disclosure are not intended to be interpreted as excluding the existence of additional embodiments that also incorporate the recited features. For example, any element or feature described in relation to an embodiment herein may be combinable with any element or feature of any other embodiment described herein, where compatible.

### Examples

1. A computer implemented method includes receiving training data comprising molecule graphs and for each molecule graph, mapping nodes of the graph via a molecule embedding model encoder to an embedding space to generate a node embedding, aggregating the node embeddings to generate molecule graph embedding vector, decoding the molecule embedding vector, via an attention based recurrent neural network decoder to generate an output including a character-based representation of the molecule, and using backpropagation of a gradient of an error from a loss function applied to the output of the decoder and a character-based representation of the input molecule graph, to modify weights of the molecule embedding model encoder and decoder for mapping of the molecule graph to the embedding space.

2. The method of example 1 wherein the loss function comprises a cross entropy of the output character-based representation and a tokenized representation of the molecule.

3. The method of example 1 wherein the loss function comprises a Connectionist temporal classification function of the output character-based representation and a tokenized representation of the molecule.

4. The method of any of examples 1-3 and further comprising initializing attention weights of the of the attention based recurrent neural network decoder.

5. The method of example 4 wherein the attention weights are initialized using a feed forward layer based on an input token and the molecule embedding vector.

6. The method of example 5 and further comprising setting a maximum decoder input length for the molecule embedding vector.

7. The method of any of examples 1-6 and further including determining multiple molecule embedding vectors for multiple molecule graphs using the embedding model having modified weights and identifying molecule graphs within a threshold distance of each other in the molecule embedding space.

8. The method of any of examples 1-7 wherein each molecule graph includes nodes and edges, wherein the embedding model comprises one or more graph neural network layers that use a message-passing framework, wherein one or more attention layers of the embedding model determine edge weights to be applied to messages received by a receiving node in the graph from one or more sending nodes in the graph, and wherein the molecule features are based in part on the edge weights and the messages.

9. The method of example 8, wherein the edge weights are based on features of the receiving node and the one or more sending nodes.

10. The method of example 9, wherein the edge weights are further based on a weighting coefficient and the one or more attention layers learn how to prioritize different messages based on the back propagation.

11. A machine-readable storage device has instructions for execution by a processor of a machine to cause the processor to perform operations to perform a method. The operations include receiving training data comprising molecule graphs and for each molecule graph, mapping nodes of the graph via a molecule embedding model encoder to an embedding space to generate node embeddings, aggregating the node embeddings to generate a molecule embedding vector, decoding the molecule embedding vector, via an attention based recurrent neural network decoder to generate an output including a character-based representation of the molecule, and using backpropagation of a gradient of an error from a loss function applied to the output of the decoder and a character-based representation of the input molecule graph to modify weights of the molecule embedding model encoder and decoder for mapping of the molecule graph to the embedding space.

12. The device of example 11 wherein the loss function comprises a cross entropy of the output character-based representation and a tokenized representation of the molecule.

13. The device of example 11 wherein the loss function comprises a Connectionist temporal classification function of the output character-based representation and a tokenized representation of the molecule.

14. The device of any of examples 11-13 and further comprising initializing attention weights of the of the attention based recurrent neural network decoder.

15. The device of example 14 wherein the attention weights are initialized using a feed forward layer based on an input token and the molecule embedding vector.

16. A computer implemented method includes receiving, at an embedding model, examples from a training data batch, wherein the examples from the training data batch includes a graph that represents a molecule and a corresponding label, outputting, from the embedding model, molecule features for each example received from the training data batch, wherein the molecule features map to an embedding space, decoding the molecule features for each example to produce a character-based representation of the molecule, calculating a loss between the produced character-based representation and the label, backpropagating gradient of the loss function to the embedding model for each example in the training data batch, and modifying learnable weights of the embedding model based on the back propagation.

17. The method of example 16, wherein the embedding model includes one or more graph neural network layers and one or more attention layers.

18. The method of example 17, wherein the graph includes nodes and edges, wherein the one or more graph neural network layers use a message-passing framework, wherein the one or more attention layers determine edge weights to be applied to messages received by a receiving node in the graph from one or more sending nodes in the graph, and wherein the molecule features are based in part on the edge weights and the messages.

19. The method of example 18, wherein the edge weights are based on features of the receiving node and the one or more sending nodes.

20. The method of example 19, wherein the edge weights are further based on a weighting coefficient and the one or more attention layers modify the weighting coefficient based on the back propagation.

Although a few embodiments have been described in detail above, other modifications are possible. For example, the logic flows depicted in the figures do not require the particular order shown, or sequential order, to achieve desirable results. Other steps may be provided, or steps may be eliminated, from the described flows, and other components may be added to, or removed from, the described systems. Other embodiments may be within the scope of the following claims.

1. A computer implemented method comprising:
receiving training data comprising molecule graphs and for each molecule graph:
mapping nodes of the graph via a molecule embedding model encoder to an embedding space to generate a node embedding;
aggregating the node embeddings to generate molecule graph embedding vector;
decoding the molecule embedding vector, via an attention based recurrent neural network decoder to generate an output including a character-based representation of the molecule; and
using backpropagation of a gradient of an error from a loss function applied to the output of the decoder and a character-based representation of the input molecule graph, to modify weights of the molecule embedding model encoder and decoder for mapping of the molecule graph to the embedding space.

2. The method of claim 1 wherein the loss function comprises a cross entropy of the output character-based representation and a tokenized representation of the molecule.

3. The method of claim 1 wherein the loss function comprises a Connectionist temporal classification function of the output character-based representation and a tokenized representation of the molecule.

4. The method of claim 1 and further comprising initializing attention weights of the of the attention based recurrent neural network decoder.

5. The method of claim 4 wherein the attention weights are initialized using a feed forward layer based on an input token and the molecule embedding vector.

6. The method of claim 5 and further comprising setting a maximum decoder input length for the molecule embedding vector.

7. The method of claim 1 and further comprising:
determining multiple molecule embedding vectors for multiple molecule graphs using the embedding model having modified weights; and
identifying molecule graphs within a threshold distance of each other in the molecule embedding space.

8. The method of claim 1 wherein each molecule graph includes nodes and edges, wherein the embedding model comprises one or more graph neural network layers that use a message-passing framework, wherein one or more attention layers of the embedding model determine edge weights to be applied to messages received by a receiving node in the graph from one or more sending nodes in the graph, and wherein the molecule features are based in part on the edge weights and the messages.

9. The method of claim 8, wherein the edge weights are based on features of the receiving node and the one or more sending nodes.

10. The method of claim 9, wherein the edge weights are further based on a weighting coefficient and the one or more attention layers learn how to prioritize different messages based on the back propagation.

11. A machine-readable storage device having instructions for execution by a processor of a machine to cause the processor to perform operations to perform a method, the operations comprising:
receiving training data comprising molecule graphs and for each molecule graph:
mapping nodes of the graph via a molecule embedding model encoder to an embedding space to generate node embeddings;
aggregating the node embeddings to generate a molecule embedding vector;
decoding the molecule embedding vector, via an attention based recurrent neural network decoder to generate an output including a character-based representation of the molecule; and
using backpropagation of a gradient of an error from a loss function applied to the output of the decoder and a character-based representation of the input molecule graph to modify weights of the molecule embedding model encoder and decoder for mapping of the molecule graph to the embedding space.

12. The device of claim 11 wherein the loss function comprises a cross entropy of the output character-based representation and a tokenized representation of the molecule.

13. The device of claim 11 wherein the loss function comprises a Connectionist temporal classification function of the output character-based representation and a tokenized representation of the molecule.

14. The device of claim 11 and further comprising initializing attention weights of the of the attention based recurrent neural network decoder.

15. The device of claim 14 wherein the attention weights are initialized using a feed forward layer based on an input token and the molecule embedding vector.

16. A computer implemented method comprising:
receiving, at an embedding model, examples from a training data batch, wherein the examples from the training data batch includes a graph that represents a molecule and a corresponding label;
outputting, from the embedding model, molecule features for each example received from the training data batch, wherein the molecule features map to an embedding space;
decoding the molecule features for each example to produce a character-based representation of the molecule;
calculating a loss between the produced character-based representation and the label;
backpropagating gradient of the loss function to the embedding model for each example in the training data batch; and
modifying learnable weights of the embedding model based on the back propagation.

17. The method of claim 16, wherein the embedding model includes one or more graph neural network layers and one or more attention layers.

18. The method of claim 17, wherein the graph includes nodes and edges, wherein the one or more graph neural network layers use a message-passing framework, wherein the one or more attention layers determine edge weights to be applied to messages received by a receiving node in the graph from one or more sending nodes in the graph, and wherein the molecule features are based in part on the edge weights and the messages.

19. The method of claim 18, wherein the edge weights are based on features of the receiving node and the one or more sending nodes.

20. The method of claim 19, wherein the edge weights are further based on a weighting coefficient and the one or more attention layers modify the weighting coefficient based on the back propagation.
